(19) United States
(12) Patent Application Publication
(10) Pub. No.: US 2026/0232622 A1
(43) Pub. Date: Aug. 13, 2026

(54) A COMBINATION OF A SOURCE OF ISOFLAVONES AND AT LEAST A NATURAL EXTRACT FOR TREATMENT, PREVENTION, OR SUPPORTING IMPROVEMENT OF COGNITIVE SYMPTOMS RELATED TO MENOPAUSE

(71) Applicant: SOCIÉTÉ DES PRODUITS NESTLÉ S.A., Vevey (CH)

(72) Inventors: Marjorie Marie-Carmen GUITARD, Savigny (CH); Ondine WALTER, Saint-Sulpice (CH); Maria ROULUND-NORGAARD, Lausanne (CH); Silvia Cristina VASQUEZ, Lausanne (CH); Susana REBER, Celigny (CH); Raphael THURN-TAXIS, Zürich (CH)

(21) Appl. No.: 19/156,082

(22) PCT Filed: Feb. 15, 2024
(86) PCT No.: PCT/EP2024/053818

§ 371 Date: Aug. 13, 2025

Related U.S. Application Data
(60) Provisional application No. 63/446,065, filed on Feb. 16, 2023.

(51) Int. Cl. A61K 31/352; A61K 36/235; A61K 36/258; A61K 36/28; A61K 36/48; A61K 36/53; A61K 36/81; A61K 36/82; A61P 25/28

(52) U.S. Cl. CPC A61K 31/352; A61K 36/235; A61K 36/258; A61K 36/28; A61K 36/48; A61K 36/53; A61K 36/81; A61K 36/82; A61P 25/28

(57) ABSTRACT

A method treats or prevents cognitive menopausal symptoms and includes administering, to a human woman having or at risk of the cognitive menopausal symptoms, an effective amount of a combination of a source of isoflavones and at least one of a natural extract for supporting cognitive performance and a natural extract for promoting calmness and reducing stress. The combination may be administered in a composition that further includes one or more of Vitamin B12, Vitamin B6, Vitamin B complex, iron, magnesium, and/or a refreshing/cooling agent. Single or multiple doses of the composition may be administered per day, the multiple doses including a first dose and a second dose. The first dose may be administered to the human woman in the morning, and the second dose may be administered to the human woman in the night.

### TECHNICAL FIELD

The present disclosure generally relates to compositions and methods that treat cognitive symptoms related to menopause. In preferred embodiments, the present disclosure relates to treating cognitive symptoms related to menopause by administering a combination of a source of isoflavones and at least one of a natural extract for supporting cognitive performance and a natural extract for promoting calmness and reducing stress to a human woman suffering from cognitive symptoms related to menopause.

### BACKGROUND

Menopause is the disruption of menstruation for at least 12 consecutive months and means loss of fertility. In the United States, the average age of menopause is 51 years. An estimated 6000 US women reach menopause each day, and with increasing life expectancy, will spend approximately 40% of their lives in the post menopause phase.

Even if menopause is a normal physiological change rather than a pathological condition, it is associated with changes in the body such as the decrease of the level of estrogen, leading to various symptoms impacting the quality of life of women. Specifically, menopause is accompanied by hot flashes and night sweats, sleep disturbances, fatigue, stress, anxiety, concentration disorders, and memory impairment leading to global cognitive impairment and loss of mental agility.

To help alleviate these menopausal symptoms, hormonal therapies are available. Because their long-term use is often associated with side effects, 65% of women in the US would not consider hormonal therapies. Additionally, long term use has been shown to result in uterine cancer, breast cancer, stroke, pulmonary thromboembolism, and various side effects.

### SUMMARY

Plant-derived phytoestrogen may be a convenient alternative to hormonal therapies for menopause treatment. Plant-derived phytoestrogen contains isoflavones, which are structurally close to estrogen and bind to the estrogen receptors to show an estrogen-like effect, thereby ameliorating menopausal symptoms such as hot flashes and night sweats.

The present disclosure provides a composition that combines isoflavones derived from a natural source with at least one additional natural extract formulated to support cognitive performance and/or to promote calmness and reduce stress.

Red clover, also known as Trifolium pratense, is a plant containing phytoestrogen and isoflavones, and various scientific studies support its positive action towards menopausal symptoms. Although red clover has been tested in combination with various ingredients to alleviate menopausal symptoms (e.g., hops, pomegranate), applicant adopted a holistic approach to address the cognitive symptoms related to menopause. One aspect of the present disclosure provides a composition that combines red clover extract with an additional plant extract. In some embodiments, the composition combines red clover with an extract from lemon balm (Melissa officinalis), which improves calmness and decreases anxiety. In some embodiments, the composition combines red clover extract with ginseng extract (Panax ginseng) which support cognitive performance.

Without being bound by any theory, the present inventors believe that the particular combination of red clover extract and at least one of lemon balm or ginseng extract is of synergistic benefit in menopausal or post-menopausal woman or a woman suffering from cognitive related menopausal symptoms.

In some embodiments, the composition further comprises at least one of Vitamin B6, Vitamin B12, Vitamin B complex, Iron or Magnesium, preferably in a total amount effective to enhance normal functioning of the nervous system, to thereby create a unique blend to improve the mental well-being and overall cognition of menopausal women. In some embodiments, a cooling/refreshing agent is added to provide a direct cooling and refreshing effect into the mouth.

In another embodiment, the present disclosure provides a method for treatment, prevention, reducing an incidence of, reducing a frequency of, and/or reducing a severity of cognitive menopausal symptoms, the method comprising administering a source of isoflavones and at least one of a natural extract for supporting cognitive performance and a natural extract for promoting calmness and reducing stress to a human menopausal woman at risk of or already suffering from the cognitive menopausal symptoms. In preferred embodiments, the combination includes red clover extract and at least one of ginseng extract or lemon balm extract and is administered in a therapeutically or prophylactically effective amount. Cognitive menopausal symptoms can include at least one of hot flashes, night sweats, sleeping disturbances, fatigue, stress, anxiety, concentration disorders, or memory impairment.

In another embodiment, the present disclosure provides a method of making a food supplement, food or beverage composition for treating, supporting improvement of, preventing, reducing an incidence of, reducing a frequency of, and/or reducing a severity of cognitive cognitive symptoms related to menopause. The method comprises adding an effective amount of a combination of a source of isoflavones and at least one of a natural extract for supporting cognitive performance and a natural extract for promoting calmness and reducing stress to at least one ingredient selected from the group consisting of protein, carbohydrate, and fat. The method can further comprise providing the food supplement/composition to a human menopausal woman at risk of or already suffering from the cognitive menopausal symptoms.

In another embodiment, the present disclosure provides a method of making a pharmaceutical composition for treating cognitive symptoms related to menopause, the method comprising adding an effective amount of a combination of a source of isoflavones and at least one of a natural extract for supporting cognitive performance and a natural extract for promoting calmness and reducing stress to at least one component selected from the group consisting of pharmaceutically acceptable carriers, diluents and excipients. The method can further comprise providing the pharmaceutical composition to a human menopausal woman at risk of or already suffering from the cognitive menopausal symptoms.

An advantage of one or more embodiments provided by the present disclosure is to improve the condition of menopausal women.

Another advantage of one or more embodiments provided by the present disclosure is to prevent or treat, support improvement of cognitive related menopausal symptoms.

Still another advantage of the present disclosure is to provide a natural remedy and support for menopausal symptoms without the side effects of conventional hormonal therapies.

Yet another advantage of one or more embodiments is to provide a synergistic effect greater than the expected effect when the red clover or another natural extract containing isoflavones, and additional plant extracts, are applied individually.

Additional features and advantages are described herein and will be apparent from the following Detailed Description.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “an extract” or “the extract” includes two or more extracts.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions and methods disclosed herein may lack any element that is not specifically disclosed herein. Thus, a disclosure of an embodiment using the term “comprising” includes a disclosure of embodiments “consisting essentially of” and “consisting of” the components or steps identified.

The terms “at least one of” and “and/or” used respectively in the context of “at least one of X and Y” and “X and/or Y” should be interpreted as “X without Y,” or “Y without X,” or “both X and Y.” Where used herein, the terms “example” and “such as,” particularly when followed by a listing of terms, are merely exemplary and illustrative and should not be deemed to be exclusive or comprehensive.

A “combination of a source of isoflavones and at least one of a natural extract for supporting cognitive performance and a natural extract for promoting calmness and reducing stress” encompasses (i) a source of isoflavones and a natural extract for supporting cognitive performance, (ii) a source of isoflavones and a natural extract for promoting calmness and reducing stress, and (iii) a source of isoflavones, a natural extract for supporting cognitive performance, and a natural extract for promoting calmness and reducing stress and (iv) a first composition comprising a combination of a source of isoflavones and a natural extract for supporting cognitive performance, administered concomitantly or sequentially with a second composition comprising a combination of a source of isoflavones and a natural extract for promoting calmness and reducing stress (e.g., as separate compositions).

As used herein, “related to,” “associated with” and “linked with” mean occurring concurrently, preferably mean caused by the same underlying condition, more preferably mean that one of the identified conditions is at least indirectly caused by the other identified condition, and most preferably mean that one of the identified conditions is directly caused by the other identified condition.

The term “cognitive symptoms” refers to symptoms and conditions self-reported by women or diagnosed during, but not limited to, their transition to menopause. Such symptoms and conditions can have a indirect or direct impact on the mental wellbeing and on the cognitive performance. Symptoms and conditions can include: night sweats, hot flashes, bad quality of sleep, fatigue, bad mood, stress, anxiety, brain fog.

The terms “food”, “food supplement”, “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet supplement.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “promote,” “improve,” “increase,” “enhance” and like terms refer to the cognitive menopausal symptoms of a menopausal human woman, after administration of the composition disclosed herein (which comprises a combination of red clover extract and at least one of ginseng extract or lemon balm extract), relative to the cognitive menopausal symptoms of a menopausal human woman, obtained by administration of a composition lacking at least one of the red clover extract or the ginseng or lemon balm extract, but otherwise identically formulated.

“Menopause” is defined as a point in time 12 months after a woman experienced her last menstruation and is the result of a natural decline in reproductive hormones. A recipient of administration who is “at risk of” cognitive menopausal symptoms is a human woman who has menopause but does not yet have cognitive menopausal symptoms. A recipient of administration who is “in need of” treatment of cognitive menopausal symptoms is a human woman who is suffering from cognitive menopausal symptoms (e.g., within one month before administration of the composition, within one week before administration of the composition, or within one day before administration of the composition). In some embodiments, the recipient of administration is a human woman who has an age of at least forty-five years, for example at least fifty years.

“Extract” means a plant or a portion thereof, which has been separated from other portions with which it naturally occurs, to create a concentrated form of one or more active ingredients of the plant.

“Red Clover” is also referred to as “Trifolium pratense” and refers to this perennial plant. As used herein, “red clover extract” comprises at least one of the phytoestrogens biochanin A, formononetin, genistein, and daidzein; preferably at least two of these phytoestrogens, more preferably at least three of these phytoestrogens, and most preferably all four of these phytoestrogens. In some embodiments, the red clover extract further comprises at least one other compound from red clover, such as other isoflavones from red clover and/or other phytoestrogens from red clover. In a particularly preferred embodiment, the red clover extract comprises one or more of the phytoestrogens noted above, in a material comprising isoflavones.

As used herein, the term “isoflavones” shall refer to the plant-based compound that mimics the action of the hormone estrogen.

“Ginseng” refers to the root of one or more Panax ginseng plants. As used herein, “ginseng extract” comprises at least one of the ginsenosides Rb1, Rb2, Rc, Rd, Re, Rf or Rg1; preferably at least two of these ginsenosides, more preferably at least three of these ginsenosides, even more preferably at least four of these ginsenosides, yet more preferably at least five or at least six of these ginsenosides, most preferably all seven of these ginsenosides. In some embodiments, the ginseng extract further comprises at least one other compound from P. ginseng, such as other ginsenosides from ginseng or P. ginseng.

“Lemon balm,” also referred to as “Melissa officinalis,” is a member of the mint family. As used herein, “lemon balm extract” comprises at least rosmarinic acid and preferably one or more of volatile compounds (e.g., geranial, neral, citronellal and geraniol), triterpenes (ursolic acid and oleanolic acid), phenolic acids additional to the rosmarinic acid (e.g., caffeic acid and chlorogenic acid), and flavonoids (quercetin, rhamnocitrin, and luteolin).

A “refreshing agent” as used herein is any agent that imparts a cooling sensation to the skin and mucous membranes of the body, particularly the mouth, nose, throat, and/or gastrointestinal tract during consumption.

As used herein, “night” refers to a time in the day after 3 pm, and “morning” refers to a time in the day before 12 pm.

The term “unit dosage form,” as used herein, refers to physically discrete units suitable as unitary dosages for human and animal subjects, each unit containing a predetermined quantity of the composition disclosed herein in an amount sufficient to produce the desired effect, in association with an acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of powder in a sachet.

As used herein, a “kit” means that the identified components are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof. A single package may be one or more containers that contain the identified components, and the one or more containers are physically associated such that they are considered a unit for manufacture, distribution, sale or use.

### Embodiments

In an aspect of the present disclosure, a composition comprises a combination of a source of isoflavones and at least one of a natural extract for supporting cognitive performance and a natural extract for promoting calmness and reducing stress. Some embodiments of the composition comprise the combination in an amount effective to (i) treat cognitive related menopause symptoms in a human menopausal woman having the cognitive related menopause symptoms or (ii) prevent cognitive related menopause symptoms in a human menopausal woman at risk of the cognitive related menopause symptoms. In another aspect, a method for treating or preventing cognitive related menopause symptoms comprises orally administering an effective amount of the composition to an individual at risk or in need thereof.

The composition can treat, prevent, reduce or delay cognitive-related menopause symptoms. Cognitive related menopause symptoms can include, but are not limited to, one or more of hot flashes, night sweats, sleeping disturbances, fatigue, stress, anxiety, concentration disorders, and memory impairment.

In some embodiments, the source of isoflavones and the at least one of the natural extract for supporting cognitive performance and the natural extract for promoting calmness and reducing stress are the main active ingredients of the composition. For example, the composition may consist essentially of the source of isoflavones and the at least one of the natural extract for supporting cognitive performance and the natural extract for promoting calmness and reducing stress.

The source of isoflavones can be from a natural plant extract rich in or containing isoflavones. In some embodiments, the source of isoflavones can be soy extract or red clover extract. The composition can comprise an effective amount of isoflavones, for example between 0.5 mg and 80 mg of isoflavones, per serving size. Specifically, the composition can comprise between 0.5 mg and 4 mg, between 4 mg and 10 mg, between 10 mg and 20 mg, between 20 mg and 30 mg, between 30 mg and 40 mg, between 40 mg and 50 mg, between 50 mg and 60 mg, between 60 mg and 70 mg, and between 70 mg and 80 mg of isoflavones per serving of the composition. For example, a unit dosage form of the composition can comprise such amounts of isoflavones, and methods can comprise administering a dose of the composition that comprises such amounts of isoflavones.

In some embodiments, the source of isoflavones is red clover extract. At least a portion of the red clover extract can be obtained commercially. Additionally or alternatively, at least a portion of the red clover extract can be obtained through extraction from red clover aerial parts. In embodiments of the composition comprising red clover extract, the composition can comprise an effective amount, for example between 5 mg and 800 mg, of red clover extract per serving size containing between 0.5 mg and 80 mg of red clover isoflavones. Specifically, the composition can comprise between 5 mg and 40 mg, between 40 mg and 50 mg, between 50 mg and 100 mg, between 100 mg and 200 mg, between 200 mg and 300 mg, between 300 mg and 400 mg, between 400 mg and 500 mg, between 500 mg and 600 mg, between 600 mg and 700 mg, and between 700 mg and 800 mg of red clover extract, per serving of the composition. For example, a unit dosage form of the composition can comprise such amounts of red clover extract, and methods can comprise administering a dose of the composition that comprises such amounts of red clover extract.

Non-limiting examples of natural extracts for promoting calmness and reducing stress include lemon balm extract, chamomile extract, fennel extract, ashwagandha extract, and combinations thereof. In some embodiments, the natural extract for promoting calmness and reducing stress comprises lemon balm extract. Lemon balm extract comprises rosmarinic acid and has been associated with calmness, reduced anxiety, and better mood. In embodiments of the composition comprising lemon balm extract, the composition can comprise at least 18 mg of rosmarinic acid, for example at least 20 mg of rosmarinic acid, or at least 22 mg of rosmarinic acid from lemon balm extract, per serving of the composition. In some embodiments, a unit dosage form of the composition can comprise such amounts of rosmarinic acid from lemon balm extract, and methods can comprise administering a dose of the composition that comprises such amounts of rosmarinic acid from lemon balm extract.

In some embodiments, the natural extract for supporting cognitive performance can include one or more of green tea extract and ginseng extract. In some embodiments, the natural extract for supporting cognitive performance comprises ginseng extract. Ginseng extract comprises ginsenosides and has been associated with increased cognitive performance, attention, and memory performance. In embodiments of the composition comprising ginseng extract, the composition can comprise at least 12 mg of ginsenosides, for example at least 14 mg of ginsenosides, or at least 16 mg of ginsenosides from the ginseng, per serving of the composition.

In some embodiments, the composition further comprises a refreshing agent. The refreshing agent may be selected from, but is not limited to, refreshing natural flavors, menthol, menthol derivative compounds, acyclic and/or cyclic carboxamides, N-substituted paramenthane carboxamides, phosphine oxides, substituted p-menthanes, menthoxypropane, alpha-keto enamine derivatives, N-substituted p-menthane carboxamide, menthyl half acid ester derivatives, cubebol, and mixtures thereof.

In some embodiments, the composition further comprises at least one of Vitamin B6, Vitamin B12, Vitamin B complex, iron or Magnesium. In such embodiments, the composition may consist essentially of (i) the source of isoflavones, (ii) the at least one of the natural extract for supporting cognitive performance and the natural extract for promoting calmness and reducing stress, (iii) the at least one of Vitamin B6, Vitamin B12, Vitamin B complex, Iron or Magnesium, and optionally (iv) any refreshing agent.

The magnesium may be one or more organic and/or inorganic physiologically acceptable compounds, for example one or more of magnesium inorganic salts (such as magnesium chloride, magnesium phosphate, magnesium sulphate, magnesium oxide, magnesium hydroxide and/or magnesium carbonate), magnesium organic salts (such as magnesium citrate, magnesium maleate or mixtures thereof), and/or organic components which comprise magnesium.

The compositions may include fiber. Non-limiting examples of suitable fibers include fructo-oligosaccharides (FOS), inulin, collagen, collagen peptides, and mixtures thereof.

The composition can be in any oral nutritional form, e.g., as a health drink, as a ready-made drink, optionally as a soft drink, including juices, coffee, tea, milk-shake, yogurt drink, smoothie or soy-based drink; in a food bar; a powder, such as in sachet form; or dispersed in foods of any sort, such as baked products, cereal bars, dairy bars, snack-foods, soups, breakfast cereals, muesli, candies, tabs, cookies, biscuits, crackers (such as rice crackers), and dairy and plant-based products.

The composition may be in the form of tablets, capsules, pastilles, a powder, or a liquid, for example. The composition may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins or the like), adsorbents, carriers, fillers, co-compounds, dispersing agents, wetting agents, processing aids (solvents), flowing agents, taste masking agents, weighting agents, jellifying agents and gel forming agents.

In some embodiments, the composition (e.g., a unit dosage form of the composition) is in the form of a gummy. Gummy sweets or gummy candies (hereinafter referred to as “gummies,” or “gummy” or “gummi” in the singular) may be prepared by moulding. Preferred ‘gummies’ are confectionery products, dietary supplement or food that exhibit, at least in part, a deformable, non-rigid, plastic, rubber, chewable and/or gelatinous consistency. Gummies may comprises one or more gelling agents (such as, pectin, gelatine), sugars or other sweetener (such as stevia), flavourings, coating and/or colorants.

In some embodiments, the composition (e.g., a unit dosage form of the composition) is a powder, for example a powder in a sachet or other package, which can be opened for reconstitution of the powder in a diluent such as water or milk and oral administration of the resultant beverage. A non-limiting example of a suitable sachet is a small disposable pouch, typically of flexible film such as cellophane or paper, preferably capable of being torn open at one or both ends, and containing one serving or one unit dosage form of the composition.

The composition preferably consists of only natural ingredients to avoid unwanted side effects with conventional hormonal therapies. The composition of the current disclosure provides treatment to individuals suffering menopausal cognitive symptoms, while avoiding bleeding, bloating, breast tenderness or enlargement, headaches, mood changes, and nausea associated with synthetic hormonal therapies.

In some embodiments, the composition is vegan. A vegan composition refers to a composition free from animal products. The composition may comprise ingredients derived mostly from plants and other natural sources.

The composition can be administered to the individual at least two days per week, at least three days per week, all seven days of the week respecting serving size; for at least one week, at least one month, at least two months, at least three months, at least six months, or even longer. In some embodiments, the composition is administered to the individual consecutively for a number of days. In an embodiment, the composition can be administered to the individual daily for at least 30, 60 or 90 consecutive days.

In some embodiments, a single dose of the composition can be administered. In some embodiments, a plurality of doses of the composition can be administered sequentially in the same day or concomitantly in the same day. For example, a first dose of a combination of the source of isoflavones and the at least one of the natural extract for supporting cognitive performance and the natural extract for promoting calmness and reducing stress can be administered at a first time in a particular day, and a second dose of a combination of the source of isoflavones and at least one of the natural extract for supporting cognitive performance and the natural extract for promoting calmness and reducing stress can be administered at a second time in the particular day (e.g., at least one hour apart from each other, at least two hours apart from each other, at least three hours apart from each other, at least four hours apart from each other, at least five hours apart from each other, at least six hours apart from each other, at least seven hours apart from each other, or at least eight hours apart from each other, preferably in the same day).

In a particularly preferred embodiment, the first dose of the composition can be administered in the morning of one or more days, the first dose comprising a combination of the source of isoflavones and the natural extract for supporting cognitive performance (optionally without the natural extract for promoting calmness and reducing stress); and the second dose of the composition can be administered in the night of the one or more days in which the first dose was administered in the morning, the second dose comprising a combination of the source of isoflavones and the natural extract for promoting calmness and reducing stress (optionally without the natural extract for supporting cognitive performance).

Another aspect of the present disclosure is a kit comprising a first unit dosage form of a first composition comprising a combination of a first source of isoflavones and a natural extract for supporting cognitive performance. The kit further comprises a second unit dosage form of a second composition comprising a combination of a source of isoflavones and a natural extract for promoting calmness and reducing stress. The kit is for administration of the second unit dosage form concomitantly or sequentially to administration of the first unit dosage form. The first and second compositions each optionally have one or more different ingredients, or amounts of ingredients, relative to the other composition (e.g., the natural extract for supporting cognitive performance in the first composition is optionally absent from the second composition, and/or the natural extract for promoting calmness and reducing stress in the second composition is optionally absent from the first composition), although in some embodiments, the first and second compositions have identical ingredients and amounts thereof.

### EXAMPLES

### Example 1: Preparation of Food Supplement in Gummies Format

The following recipes were used for the preparation of food supplement gummies (Table 1):

TABLE 1

| Composition | | | | |
|---|---|---|---|---|
| (2 gummies) | Sample 1 | | Sample 2 | |
| Carbohydrate | 5 | g | 5 | g |
| Vitamin B6 | 1.7 | mg | 1.7 | mg |
| Vitamin B12 | 2.4 | mcg | 2.4 | mcg |
| Magnesium | 63 | mg | 63 | mg |
| Sodium | 10 | mg | 10 | mg |
| Herbal Blend 1: Lemon Balm | 340 | mg | — | |
| and Red Clover Extracts | | | | |

| | | | |
|---|---|---|---|
| Herbal Blend 2: Panax | — | 340 | mg |

Ginseng and Red Clover
Extracts

Other ingredients have been included in the preparation as follows: Fructooligosaccharides, Water, Pectin, Agar Agar, Tapioca Starch, Natural Flavors, Natural color, Citric Acid, Trisodium Citrate, Carnauba Wax.

For the preparation of the above-mentioned food supplement in gummies format, a mixture of fructooligosaccharides, pectin, water, tapioca starch, agar agar, and magnesium are blended until it forms a smooth and consistent mixture. The herbal blend is then added, followed by the incorporation of color, flavor, and vitamins. Once the desired pH and brix levels are achieved, citric acid is introduced, and the mixture is disposed into starch molds. The gummies are left to dry until they reach the desired brix level, after which they are removed from the molds and coated with carnauba wax.

### Example 2: Efficacy Study

Samples 1 and 2, as prepared in Example 1, were tested for a duration of 30 days by women in the United States experiencing cognitive symptoms associated with menopause (referred to as participants). Each participant tested either Sample 1 (total of 80 participants) or Sample 2 (total of 79 participants) and expressed willingness to consume the recommended dosage of 2 gummies per day for the 30-day period.

In the efficacy study, the assessment of Samples 1 and 2 was conducted through a post-trial survey evaluating the following:
a) The efficacy of the Samples in alleviating menopause symptoms was rated on a scale of 1 to 5, ranging from “Not at all efficient”, “Slightly efficient”, “Somewhat efficient”, “Very efficient” to “Extremely efficient”.
b) The efficacy of the Samples compared to previously used solutions was rated on a scale of 1 to 5, ranging from “Much less efficient”, “Less efficient”, “About the same efficiency”, “More efficient” to “Much more efficient”.

The results for the element evaluated in section a) are as follows (Table 2):

TABLE 2

| Rating | Sample 1 | Sample 2 |
|---|---|---|
| Not at all efficient | 16.25% | 21.79% |
| Slightly efficient | 13.75% | 11.54% |
| Somewhat efficient | 13.75% | 20.51% |
| Very efficient | 36.25% | 28.21% |
| Extremely efficient | 20% | 17.95% |

It can be observed from Table 2 that a large portion of the participants perceived the samples as ‘extremely efficient’ or ‘very efficient’ in reducing their menopause symptoms (56.25% for Sample 1 and 46.16% for Sample 2).

The results for the element evaluated in section b) are as follows (Table 3):

TABLE 3

| Rating | Sample 1 | Sample 2 |
|---|---|---|
| Much less efficient | 5% | 10.26% |
| Less efficient | 10% | 8.97% |
| About the same efficiency | 22.5% | 25.64% |
| More efficient | 38.75% | 35.9% |
| Much more efficient | 23.75% | 19.23% |

It can be observed from Table 3 that more than half of the participants perceived the samples as ‘much more efficient’ or ‘more efficient’ than other solutions in reducing their menopause symptoms (62.5% for Sample 1 and 55.13% for Sample 2).

1: A composition comprising a combination of a source of isoflavones and at least one of a natural extract for supporting cognitive performance and a natural extract for promoting calmness and reducing stress.

2: The composition of claim 1, wherein the composition is in a unit dosage form that comprises an amount of the composition effective for treatment or prevention of cognitive menopausal symptoms.

3: The composition of claim 1, wherein the composition is vegan.

4: The composition of claim 1, wherein the source of isoflavones is selected from the group consisting of soy extract, red clover extract, and combinations thereof.

5: The composition of claim 1, wherein the natural extract for promoting calmness and reducing stress is selected from the group consisting of lemon balm extract, chamomile extract, fennel extract, ashwaganda extract and combinations thereof.

6: The composition of claim 1, wherein the natural extract for supporting cognitive performance is selected from the group consisting of ginseng extract and green tea extract.

7: The composition of claim 1, further comprising an additional ingredient selected from the group consisting of inulin, collagen, collagen peptides, fructooligosaccharides, and combinations thereof.

8: A method for treatment or prevention of cognitive menopausal symptoms, the method comprising administering to a human woman in need thereof or at risk thereof an effective amount of a combination of a source of isoflavones and at least one of a natural extract for supporting cognitive performance and a natural extract for promoting calmness and reducing stress.

9: The method of claim 8, wherein the source of isoflavones comprises red clover extract.

10: The method of claim 8, wherein the combination comprises a natural extract for supporting cognitive performance, wherein the natural extract for supporting cognitive performance comprises ginseng extract and is administered to the human woman in need thereof or at risk thereof in a dose comprising an amount of the ginseng extract comprising at least 12 mg ginsenosides.

11: The method of claim 8, wherein the combination comprises a natural extract for promoting calmness and reducing stress, wherein the natural extract for promoting calmness and reducing stress comprises lemon balm extract and is administered to the human woman in need thereof or at risk thereof in a dose comprising an amount of the lemon balm extract comprising at least at least 18 mg rosmarinic acid.

12: The method of claim 8, wherein the combination is administered in a composition that further comprises one or more vitamins and minerals selected from the group consisting of vitamin B12, vitamin B6, magnesium, and combinations thereof.

13: The method of claim 8, wherein the cognitive menopausal symptoms are selected from the group consisting of hot flashes, night sweats, sleeping disturbances, fatigue, stress, anxiety, concentration disorders, memory impairment and combinations thereof.

14: The method of claim 8, wherein the combination is administered in a composition that further comprises a refreshing agent.

15: The method of claim 8, wherein a single dose of the combination is administered per day.

16: The method of claim 8, wherein a plurality of doses of the combination are administered per day, the plurality of doses comprising a first dose in which the combination comprises a first source of isoflavones and a natural extract for supporting cognitive performance, the plurality of doses further comprising a second dose in which the combination comprises a second source of isoflavones and a natural extract for promoting calmness and reducing stress, and the second dose is administered concomitantly or sequentially to the first dose.

17: The method of claim 8, wherein a plurality of doses of the combination are administered per day, the plurality of doses comprising a first dose in which the combination comprises red clover extract and ginseng extract, the plurality of doses further comprising a second dose in which the combination comprises red clover extract and lemon balm extract, and the second dose is administered concomitantly or sequentially to the first dose.

18: The method of claim 16, wherein the first dose is administered to the human woman in need thereof or at risk thereof in the morning of one of more days, and the second dose is administered to the human woman in need thereof or at risk thereof in the night of the one of more days.

19: The method of claim 8, wherein the combination is administered to the human woman in need thereof or at risk thereof in a gummy.

20: A composition comprising a combination of red clover extract and at least one of ginseng extract or lemon balm extract.

21: The composition of claim 20, wherein the composition is in a unit dosage form that comprises an amount of the composition effective for treatment or prevention of cognitive menopausal symptoms.

22: The composition of claim 20, wherein the composition is vegan.

23: The composition of claim 20, further comprising an additional ingredient selected from the group consisting of inulin, collagen, collagen peptides, fructooligosaccharides, and combinations thereof.

24: A method of making a food or a food supplement composition for treating or preventing cognitive symptoms related to menopause, the method comprising adding an effective amount of a combination of red clover extract and at least one of ginseng extract or lemon balm extract to at least one ingredient selected from the group consisting of protein, carbohydrate, and fat.

25: The method of claim 24, further comprising providing the food composition to a human menopausal woman at risk of or already suffering from the cognitive symptoms related to menopause.

26: A kit comprising a first unit dosage form of a first composition comprising a combination of a source of isoflavones and a natural extract for supporting cognitive performance, the kit further comprising a second unit dosage form of a second composition comprising a combination of a source of isoflavones and a natural extract for promoting calmness and reducing stress, for administration of the second unit dosage form concomitantly or sequentially to administration of the first unit dosage form, wherein the first and second compositions each optionally have one or more different ingredients relative to the other composition.
